(19) United States
(12) Patent Application Publication
(10) Pub. No.: US 2006/0014776 A1
(43) Pub. Date: Jan. 19, 2006

(54) Antitumoral compounds

(75) Inventors: Antonio Fernandez, Leon (ES); Jesus A. De La Fuente, Madrid (ES); Sonia Manzanaro, Madrid (ES)

Correspondence Address: FISH & RICHARDSON PC, P.O. BOX 1022, MINNEAPOLIS, MN, 55440-1022, US

(73) Assignee: Instituto Biomar S.A., Madrid (ES)

(21) Appl. No.: 10/893,464

(22) Filed: Jul. 16, 2004

(51) Int. Cl. A61K 31/4745; C07D 471/02

(52) U.S. Cl. 514/291; 546/81

(57) ABSTRACT

Compounds of general formula I or a pharmaceutically acceptable salts, derivatives or prodrugs thereof are of use in treatment of cancer; 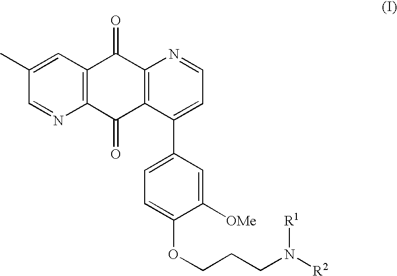
wherein R¹ and R² are independently selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, C₂-C₁₂ alkenylidene, substituted or unsubstituted C₃-C₁₂ alkynylidene and substituted or unsubstituted C₂-C₁₂ acyl.

### FIELD OF THE INVENTION

The present invention relates to new antitumoral compounds, pharmaceutical compositions containing them and their use as antitumoral agents.

### BACKGROUND OF THE INVENTION

Products containing an anthracene-9,10-dione substructure are an important class of antitumour compounds. They include the anthracyclines (Lown, J. W. Chem. Soc. Rev. 1993, 22, 165. Sengupta, S. K., in Foye, W. O. (Ed.). Cancer Chemotherapeutic Agents, chapter 5. American Chemical Society, 1995) as mitoxantrone, the pluramycins (Abe, N.; Enoki, N.; Nakakita, Y.; Uchida, H.; Nakamura, T.; Munekata, M. J. Antibiot. 1993, 46, 1536. Hansen, M.; Hurley, L. J. Am. Chem. Soc. 1995, 117, 2421) and some of the enediyne antibiotics (Konishi, M.; Ohkuma, H.; Tsuno, T.; Oki, T.; Van Duyne, G. D.; Clardy, J. J. Am. Chem. Soc. 1990, 112, 3715. Nicolau, K. C.; Dai, W.-M.; Hong, Y. P.; Tsay, S.-C.; Baldridge, K. K.; Siegel, J. S. J. Am. Chem. Soc. 1993, 115, 7944). At least in the case of the anthracyclines, the antitumour activity is mainly attributed to two mechanism of action. Because of their planar chromophore, they are able to insert between two base pairs in the DNA helix (Cashman, D. J.; Kellogg, G. E. J. Med. Chem. 2004, 47, 1360-1374) causing a local untwisting of the helix that results in topoisomerase II inhibition (Gewirtz, D. A. Biochem. Pharmacol. 1999, 57, 727-741), miscoding and possible cell death (Pan, S.-S; Pedersen, L.; Bachur, N. R.; Mol. Pharmacol. 1981, 19, 184. Hertzberg, R. P.; Dervan, P. B. Biochemistry 1984, 23, 3934. Kapuscinski, J.; Darzynkiewicz, Z.; Traganos, F.; Melamed, M. R. Biochem Pharmacol. 1981, 30, 231. Gatto, B.; Zagotto, G.; Sissi, C.; Cera, C.; Uriarte, E. J. Med. Chem. 1996, 39, 3114. Barasch, D.; Zipori, O.; Ringel, I.; Ginsburg, I.; Samuni, A.; Katzhendler J. Eur. J. Med. Chem. 1999, 34, 597). Additionally, their antitumour activity may be associated to the formation of DNA damaging anion-radical intermediates by reduction of the quinone unit (Taatjes, D. J.; Koch, T. H. Curr. Med. Chem. 2001, 8, 15-29.). The generation of reactive oxygen species also has a role in modulation of angiogenesis by the anthracyclines (Wakabayashi, I.; Groscher, K. Curr. Med. Chem. 2003, 10, 427-436).

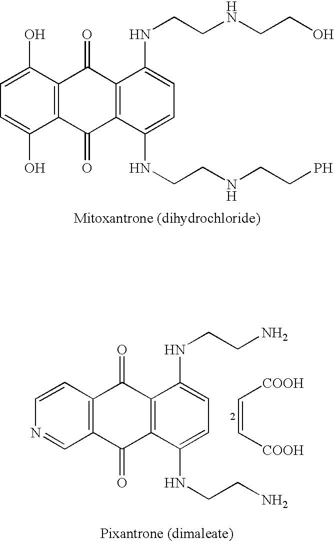

Isosteric substitution of one or more carbons of the benzene rings by nitrogen atoms should afford compounds with geometries similar to those of the parent compounds, but with increased affinity for DNA due to the presence of sites suitable for hydrogen bonding or ionic interactions. Also, the electron-withdrawing properties of the heterocyclic rings would facilitate the formation of anion-radicals. Based on this idea, some aza bioisosteres related to the anthracene-9,10-diones have been synthesized and screened in vitro and in vivo against a wide spectrum of tumour cell lines (Krapcho, A. P.; Maresch, M. J.; Hacker, M. P.; Hazelhurst, L.; Menta, E.; Oliva, A.; Spinelli, S.; Beggiolin, G.; Giuliani, F. C.; Pezzoni, G.; Tignella, S. Curr. Med. Chem. 1995, 2, 803). Among them, pixantrone shows promise for development as anticancer agent, currently being in phase III trials for the treatment of non-Hodgking's lymphoma (Borchmann, P.; Reiser, M. IDrugs 2003, 6, 486). Interestingly, this compound showed an antitumour activity comparable to that of mitoxantrone and doxorubicin but it showed no measurable cardiotoxicity compared to them at equi-effective doses in animal models (Beggiolin, G.; Crippa, L.; Menta, E.; Manzotti, C.; Cavalletti, E.; Pezzoni, G.; Torriani, D.; Randisi, E.; Cavagnoli, R.; Sala, R.; Giulinani, F. C.; Spinelli, S. Tumori 2001, 6, 407).

Although the considerations outlined above regarding bioisosteric replacement of carbon by nitrogen would apply particularly well to diaza derivatives, these compounds have received little attention and most of the published studies are focused on 1,8-diazaanthraquinones (Nebois, P.; do Nascimento, S. C.; Boitard, M.; Bartoli, M. H.; Fillion, H. Pharmazie 1994, 49, 819. Ramos, M. T.; Diaz-Guerra, L. M.; Garcia-Copin, S.; Avendaño, C. Il Farmaco 1996, 51, 375. Lee, H.; Lee, S. I.; Yang, S. I. Bioorg. Med. Chem. Lett. 1998, 8, 2991. Lee, H.; Lee, S.-I.; Cho, J.; Choi, S. U. Yang, S.-I. E. Eur J. Med. Chem. 2003, 38, 695. Delfourne, E.; Darro, F.; Bontemps-Subielos, N.; Decaestecker, C.; Bastide, J.; Frydman, A.; Kiss, R. J. Med. Chem. 2001, 44, 3275). 1,5-Diazaanthraquinones have been mainly used as intermediates in the synthesis of pyridoacridines (Delfourne, E.; Kiss, R.; Le Corre, L.; Dujols, F.; Bastide, J.; Collignon, F.; Lesur, B.; Frydman, A.; Darro, F. J. Med. Chem. 2003, 46, 3536. Kitahara, Y.; Tamura, F.; Nishimura, M.; Kubo, A. Tetrahedron 1998, 54, 8421. Kitahara, Y.; Nakahara, S.; Yonezawa, T.; Nagatsu, M.; Shibano, Y.; Kubo, A. Tetrahedron 1997, 53, 17029) but their potential as antitumour agents is almost unexplored (De la Fuente, J. A.; Martin, M. J.; Blanco, M. M.; Pascual-Alfonso, E.; Avendaño, C.; Menendez, J. C. Bioorg. Med. Chem. Lett. 2001, 9, 1807. WO9959996). Most of these 1,5-Diazaanthraquinones have not progressed due to formulation problems.

Cancer is a leading cause of death in animals and humans. Several efforts have been and are still being undertaken in order to obtain an antitumour agent active and safe to be administered to patients suffering from a cancer. The problem to be solved by the present invention is to provide compounds that are useful in the treatment of cancer.

### SUMMARY OF THE INVENTION

In one aspect, the present invention is directed to compounds of general formula I or a pharmaceutically acceptable salt, derivative or prodrug thereof 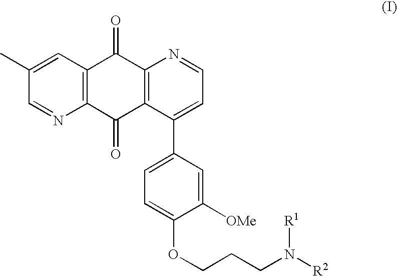
wherein R¹ and R² are independently selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₁-C₁₂ alkylidene, substituted or unsubstituted C₂-C₁₂ alkenylidene, substituted or unsubstituted C₃-C₁₂ alkynylidene and substituted or unsubstituted C₂-C₁₂ acyl.

In another aspect, the present invention is also directed to the use of compounds of formula I or pharmaceutically acceptable salts, derivatives or prodrugs thereof in the treatment of cancer, or in the preparation of a medicament for the treatment of cancer.

The present invention also relates to the obtention of the compounds of formula I from synthetic procedures.

In another aspect, the present invention is directed to pharmaceutical compositions containing a compound of formula I or pharmaceutically acceptable salts, derivatives or prodrugs thereof together with a pharmaceutically acceptable carrier or diluent.

### DETAILED DESCRIPTION OF PREFERRED EMBODIMENTS

The present invention relates to compounds of general formula I as defined above.

In these compounds the substituents can be selected in accordance with the following guidance:

Alkyl group preferably have from 1 to 12 carbon atoms. One more preferred class of alkyl group has from 1 to about 6 carbon atoms, and most preferably 1, 2, 3 or 4 carbon atoms. Methyl, ethyl and propyl including isopropyl are particularly preferred alkyl groups in the compounds of the present invention. Another more preferred class of alkyl group has from 4 to about 12 carbon atoms, yet more preferably from 5 to about 8 carbon atoms, and most preferably 5, 6, 7 or 8 carbon atoms. Pentyl, hexyl, heptyl or octyl are particularly preferred alkyl groups in the compounds of the present invention. As used herein, the term alkyl, unless otherwise modified, refers to both cyclic and noncyclic groups, although cyclic groups will comprise at least three carbon ring members.

Preferred alkenyl and alkynyl groups in the compounds of the present invention have one or more unsaturated linkages and from 2 to about 12 carbon atoms. One more preferred class of alkenyl or alkynyl groups has from 2 to about 6 carbon atoms, and most preferably 2, 3 or 4 carbon atoms. Another more preferred class of alkenyl or alkynyl groups has from 4 to about 12 carbon atoms, yet more preferably from 5 to about 8 carbon atoms, and most preferably 5, 6, 7 or 8 carbon atoms. The terms alkenyl and alkynyl as used herein refer to both cyclic and noncyclic groups.

Alkylidene groups may be substituted or unsubstituted and preferably have from 1 to 12 carbon atoms. One more preferred class of alkylidene groups has from 1 to about 8 carbon atoms, yet more preferably from 1 to about 6 carbons atoms, and most preferably 1, 2, 3 or 4 carbon atoms. Methylidene, ethylidene and propylidene including isopropylidene are particularly preferred alkylidene groups in the compounds of the present invention.

Alkenylidene groups in the compounds of the present invention may be substituted or unsubstituted, have one or more unsaturated linkages and from 2 to about 12 carbon atoms, more preferably from 2 to about 8 carbon atoms, still more preferably from 2 to about 6 carbons atoms, and even more preferably 2, 3 or 4 carbon atoms.

Preferred alkynylidene groups in the compounds of the present invention may be substituted or unsubstituted, have one or more unsaturated linkages and from 3 to about 12 carbon atoms, more preferably from 3 to about 8 carbon atoms, still more preferably from 3 to about 6 carbons atoms, and even more preferably 3 or 4 carbon atoms.

An acyl group is of the form RCO— wherein R is an organic group such as an alkyl group. Suitable acyl groups have from 2 to about 12 carbon atoms, more preferably from 2 to about 8 carbon atoms, still more preferably from 2 to about 6 carbon atoms, even more preferably 2 carbon atoms. Other acyl groups can be employed, including for example arylalkylacyl.

The groups above mentioned may be substituted at one or more available positions by one or more suitable groups such as OR′, ═O, SR′, SOR′, SO₂R′, 259 NO₂, NHR′, N(R′)₂, ═N—R′, NHCOR′, N(COR′)₂, NHSO₂R′, CN, halogen, C(═O)R′, CO₂R′, OC(═O)R′ wherein each of the R′ groups is independently selected from the group consisting of H, OH, NO₂, NH₂, SH, CN, halogen, ═O, C(═O)H, C(═O)CH₃, CO₂H, substituted or unsubstituted C₁-C₁₂ alkyl, substituted or unsubstituted C₂-C₁₂ alkenyl, substituted or unsubstituted C₂-C₁₂ alkynyl and substituted or unsubstituted aryl.

The term “pharmaceutically acceptable salts, derivatives, prodrugs” refers to any pharmaceutically acceptable salt, ester, solvate, hydrate or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline form either as free compounds or as solvates (e.g. hydrates) and it is intended that both forms are within the scope of the present invention. Methods of solvation are generally known within the art.

Any compound that is a prodrug of a compound of formula I is within the scope and spirit of the invention. The term “prodrug” is used in its broadest sense and encompasses those derivatives that are converted in vivo to the compounds of the invention. Such derivatives would readily occur to those skilled in the art.

Preferred compounds of the invention and of formula I are those of general formula II 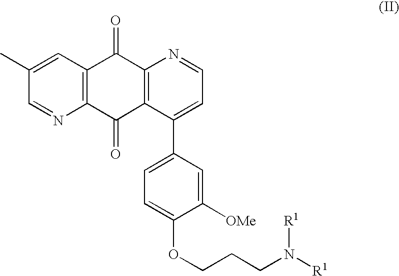
wherein R¹ group has the same meaning defined above.

Particularly preferred R¹ is H and C₁-C₁₂ alkyl.

Particularly preferred compounds of the invention are those of general formula III 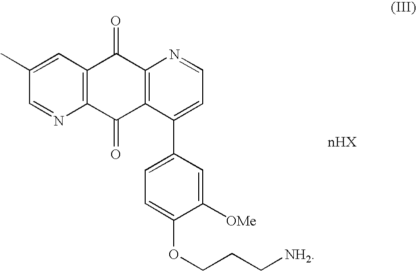
wherein X is selected from the group consisting of F, Cl, Br and I.

Particularly preferred compound of the invention is IV.

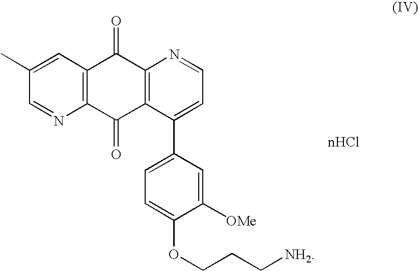

Compound of formula I are preferably obtained by synthetic procedures. In a preferred route, the process comprises reacting an N-protected {3-[2-methoxy-4-(4,4,5,5-tetramethyl-[1,3,2]dioxaborolan-2-yl)-phenoxyl]-propyl}-carbamic acid of formula: 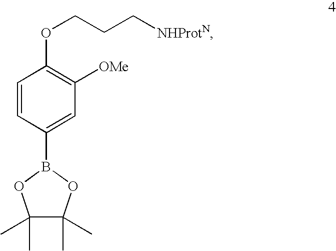
where Prot^{N} is a protecting group, with a 9-halo-3-methyl-pyrido[2,3-g]quinoline-5,10-dione of formula: 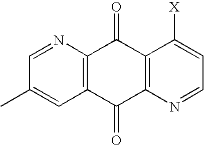
to give a protected form of a compound of this invention of formula:

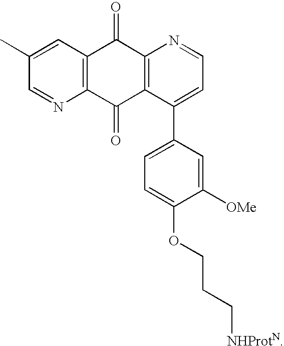

The protection can then be removed. Before or after removing N-protection, substituents can be introduced on the terminal amino group. The 9-halo-3-methyl-pyrido[2,3-g]quinoline-5,10-dione can be prepared from a 4,6-dihalo-5,8-quinolinequinone. Preferred halo for the synthesis is bromo.

### SUMMARY OF THE DRAWINGS

FIG. 1 provides data from the clonogenic assay with compound IV.

FIGS. 2 and 3 relate to an in vivo study with compound IV, 4 mice were in the control group, 4 in the 2 mg/ml and 1 in the 3 mg/ml groups.

### EXAMPLES

### Synthesis of Compound IV

Compound IV can be obtained as follow:

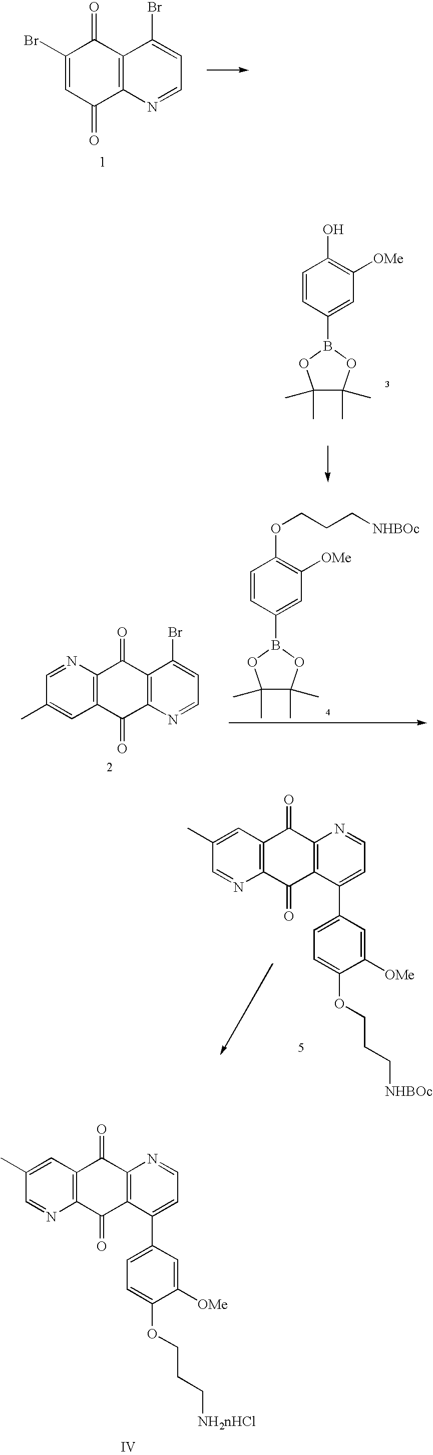

### Synthesis of 9-bromo-3-methyl-pyrido[2,3-g]quinoline-5,10-dione

To a solution of the known 4,6-dibromo-5,8-quinolinequinone (Angel de la Fuente J, Jesus Martin M, del Mar Blanco M, Pascual-Alfonso E, Avendano C, Carlos Menendez J. Bioorg. Med. Chem. 2001, 9, 1807-1814) (500 mg, 1.57 mmol) in 1,2-dichloroethane (13 mL) at 80° C., methacrolein dimethylhydrazone (235 mg, 2.01 mmol) in 1,2-dichroroethane (4 mL) was dropwise added. The mixture was kept at 80° C. for 30 min, then cooled to room temperature and the solvent was evaporated. The crude was purified by silica gel column chromatography (AcOEt) affording 9-bromo-3-methyl-pyrido[2,3-g]quinoline-5,10-dione (332 mg, 68%) as a brown solid. (Found: C, 51.53; H, 2.30; N, 9.27. C₁₃H₇BrN₂O₂ requires: C, 51.51; H, 2.33; N, 9.24); mp >200° C. (descomp.); υ (KBr) 1687 cm⁻¹; δ_{H} (CDCl_{3,} 300 MHz): 8.96 (d, 1H, J=2.2 Hz), 8.76 (d, 1H, J=5.0 Hz), 8.45 (d, 1H, J=2.2 Hz), 8.00 (d, 1H, J=5.0 Hz), 2.57 (s, 3H); δ_{C} (CDCl_{3,} 75 MHz): 180.72, 179.71, 156.97, 153.65, 150.39, 146.58, 139.73, 135.31, 135.25, 134.61, 129.09, 129.02, 19.15; MS (APCI-positive) m/z: 304 [M+H]⁺.

### Synthesis of {3-[2-methoxy-4-(4,4,5,5-tetramethyl-[1,3,2]dioxaborolan-2-yl)-phenoxyl]-propyl}-carbamic acid tert-butyl ester

A suspension of 2-methoxy-4-(4,4,5,5-tetramethyl-[1,3,2]dioxaborolan-2-yl)-phenol (500 mg, 2.00 mmol), (3-bromo-propyl)-carbamic acid ter-butyl ester (714 mg, 3.00 mmol), Cs₂CO₃ (1.06 g, 3.00 mmol), a catalytic amount of KI and Bu₄NI in anhydrous DMF (10 mL) was heated at 55-60° C. for 18 h. The mixture was cooled to room temperature, poured into H₂O and extracted with Et₂O. The combined organic layers were washed with NaCl, dried (Na₂SO₄). and evaporated under reduced pressure. The crude was purified by silica gel column chromatography (hexane:EtOAc; 3:1) obtaining {3-[2-methoxy-4-(4,4,5,5-tetramethyl-[1,3,2]dioxaborolan-2-yl)-phenoxy]-propyl}-carbamic acid tert-butyl ester (652 mg, 80%) as a pale yellow solid. Mp-86.7-88.2° C.; ¹H-NMR (CDCl₃) δ 7.39 (dd, 1H, J=8.0 Hz, J=1.1 Hz), 7.28, (d, 1H, J=1.1 Hz), 6.86 (d, 1H, J=8.0 Hz), 5.62 (sa, 1H), 4.12 (t, 2H, J=5.6 Hz), 3.92 (m, 2H), 3.38 (s, 3H), 2.01 (m, 2H), 1.45 (s, 9H), 1.34 (s, 12H); ¹³C-NMR (CDCl₃) δ 156.38, 151.06, 148.95, 128.72, 117.04, 112.14, 83.89, 79.06, 68.31, 55.98, 39.29, 29.44, 28.71, 25.06; MS (ESI-positive) m/z: 308 [M-Boc]⁺, 430 [M+Na]⁺.

### Synthesis of {3-[2-methoxy-4-(8-methyl-5,10-dioxo-5,10-dihydro-pyrido[2,3-g]quinolin-4-yl)-phenoxy]-propyl}-carbamic acid tert-butyl ester

To a solution of 9-bromo-3-methyl-pyrido[2,3-g]quinoline-5,10-dione (1 mmol), Pd(PPh₃)₄ (ca. 5% mol), Na₂CO₃ (2M) (4 mmol) in toluene (25 mL) y EtOH (1.2 mL) was added {3-[2-methoxy-4-(4,4,5,5-tetramethyl-[1,3,2]dioxaborolan-2-yl)-phenoxy]-propyl}-carbamic acid tert-butyl ester (1.1 mmol). The mixture was heated at 80° C. for 48 h and then cooled to rt. The reaction was diluted with and washed with H₂O. The aqueous phase was extracted with CH₂Cl₂ and the combined organic layers were washed with brine, dried (Na₂SO₄) and evaporated under reduced pressure. The resulting crude was purified by cromatography on silice gel column (EtOAc) affording {3-[2-methoxy-4-(8-methyl-5,10-dioxo-5,10-dihydro-pyrido[2,3-g]quinolin-4-yl)-phenoxy]-propyl}-carbamic acid tert-butyl ester (72%) as a solid.

Synthesis of Compound IV

{3-[2-Methoxy-4-(8-methyl-5,10-dioxo-5,10-dihydro-pyrido[2,3-g]quinolin-4-yl)-phenoxy]-propyl}-carbamic acid tert-butyl ester (106 mg, 0.198 mmol) was treated with 38 mL of HCl 7.5 M in 1,4-dioxane at room temperature for 30 min. The precipitate was filtered and washed with 1,4-dioxane. This precipitate was recrystallized from EtOH to get compound IV (64 mg, 70%). Mp→250° C. (decomp.); ¹H-NMR (Methanol-d₄) δ 9.00 (d, 1H, J=5.1 Hz), 8.92 (s, 1H), 8.61 (s, 1H), 7.81 (d, 1H, J=5.1 Hz), 7.14 (d, 1H, J=1.9 Hz), 7.12 (d, 1H, J=8.1 Hz), 7.06 (dd, 1H, J=8.1, 1.9 Hz), 4.29 (t, 2H, J=6.1 Hz), 3.88 (s, 3H), 3.29 (t, 2H, J=6.1 Hz), 2.63 (s, 3H), 2.25 (qt, 2H, J=6.1 Hz).; ¹³C-NMR (Methanol-d₄) δ 182.09, 182.01, 156.33, 154.96, 153.32, 150.45, 150.16, 149.94, 147.76, 141.60, 136.85, 133.53, 132.60, 130.45, 130.01, 122.28, 114.40, 113.71, 68.76, 56.58, 39.56, 28.06, 18.76; MS (ESI-positive) m/z:344 [M+H]⁺.

Bioasays for Antitumor Screening

Disk Diffusion Soft Agar Colony Formation Assay

An in vitro cell-based assay was employed to identify solid tumor selectivity for compound IV. The differential cytotoxicity (Valeriote, F.; Grieshaber, C. K.; Media, J.; Pietraszkiewicz, H.; Hoffman, J.; Pan, M.; McLaughlin, S. J. Exp. Ther. Oncol. 2002, 2, 228-236) is expressed by observing a zone differential between any solid tumor cell (Colon38, ColonH116, Lung H125) and either leukemia cells (L1210 or CEM) or normal cells (CFU-GM). A sample is designated as “solid tumor selective” if (zone units of solid tumor—normal cells or leukemia cells) is greater than 250 units.

Preparation of Cell Suspensions:

Colon 38 gives a good monodispersed cell suspension with mechanical disruption from a mouse tumor. Colon 38 (approx. 1 g) is cut into small fragments in 15 ml of Hank's Balanced Salt Solution (HBSS) over a 100-mesh sieve and gently forced through by the scissors with HBSS constantly perfusing the sieve. The material is then drawn into and out of a 5 ml syringe without a needle to further disperse the cell clumps. It is then diluted and plated in 0.3% agarose in DMEM plus 10% heat-inactivated Bovine Calf Serum (BCS). For plating of all of the cell types other than the normal CFU, the 60 mm plates are first prepared with a hard agar bottom layer (0.6% agar in RPMI-1640 plus 15% BCS).

All of the human cancer cell lines are maintained in cell culture. They are removed from their cultures by a trypsin-collagenase-DNAase cocktail. Their plating efficiencies are sufficiently high that 30,000 to 60,000 cells in 3 ml produce the desired number of colonies (over 10,000 per plate) in the 60 mm plates. This soft agar top layer (0.3% with the serum and media as above) plus the titrated tumor cells are poured into the plates and allowed to solidify. For CFU-GM, the femoral marrow of C57B1/6 mice is flushed with MEM-alpha; 2 mL per femur. The cells are passed through an 18-gauge needle twice and the monodispersed suspension counted. A total of 1.5×10⁶ cells are plated in 3 ml of 0.3% agar with the addition of 10% L-cell conditioned media, which provides colony stimulating factor, in MEM-alpha plus 10% BCS. For human CFU-GM, the cells are obtained from Poietic Technologies, Inc. (Gaithersburg, Md.) overnight and washed twice with PBS before being titered and added to the agar mixture. The same cell number, culture conditions and conditioning factors are used as with the murine marrow.

Sample Preparation:

The compounds are solubilized in between 0.25 and 1 ml DMSO.

Zone Assay Methodology:

A volume of 15 μl of each sample is dropped onto a 6.5 mm disk (Baxter filter disks). The disk is allowed to dry overnight and then placed close to the edge of the petri dish. The plates are incubated for 7-10 days (depending upon the cell type) and examined by an inverted stereo-microscope (10×) for measurement of the zone of inhibition measured from the edge of the filter disk to the beginning of normal-sized colony formation. The diameter of the filter disk, 6.5 mm, is arbitrarily taken as 200 units. A zone of less than 300 units is taken as the extract is of insufficient activity to be of further interest. A difference in zones between solid tumor cells and either normal or leukemia cells of 250 units defines solid tumor selective compounds. If the test material is excessively toxic at the first dosage, we then retest a range of dilutions of the agents (at either 1:4 or 1:10 decrements) against the same tumors. At some dilution, quantifiable cytotoxicity is invariably obtained.

The activity results for compound IV appear in Table 1:

TABLE 1
Zone Unit Differentials in the Disk Diffusion Soft
Agar Colony Formation Assay^{a} for compound IV

| | | | | HUMAN | | | |
|---|---|---|---|---|---|---|---|
| CONC. | MURINE | | | | | | CFU- |
| (μg/ml) | L1210 | C38 | CFU-GMm | H116 | H125 | CEM | GMh |
| 7.5 | 500 | 550 | 650 | 800 | >1000 | 550 | 650 |
| 1.9 | ND | 100 | ND | 350 | 600 | 150 | ND |

a Measured in zone units: 200 zone units=6 mm. Murine cell lines: L1210 (lymphocytic leukemia), C38 (colon adenocarcinoma), CFU-GM-m (colony-forming unit-granulocyte macrophage; normal hematopoietic). Human cell lines: H116 (colon), H125 (lung), CEM (leukemia), CFU-GM-h (colony-forming unit-granulocyte macrophage; normal hematopoietic).b Solvent partition fraction codes are outlined in the Experimental Section.*ND=not determined as the zone units for H116, H125, or C38 were <250.

Clonogenic Assay

Concentration- and time-survival studies will be carried out against HCT-116 cells. HCT-116 cells are seeded at 200 to 20,000 cells in 60 mm dishes. Drug is added to the medium (RPMI+10% FBS) at concentrations of 1 mg/ml and 10-fold dilutions thereof. At either 2 hr or 24 hr, the drug-containing media is removed and fresh media without drug is added. The dishes are incubated for 7 days, media removed and the colonies stained with methylene blue. Colonies containing 50 cells or more are counted. The results are normalized to an untreated control. Plating efficiency for the untreated cells is about 90%. Repeat experiments are carried out to define the cell survival range between 100% and 10^{-'}survival. Subsequently, a concentration is chosen that yields a survival of 10⁻² at 24 hr and time-survival studies carried out from 0 to 48 hr. Both concentration- and time-survival curves are constructed so that AUC values can be calculated. A comparison of tumor to normal provides a theoretical basis for determining whether a compound might be active in therapeutic trials in vivo.

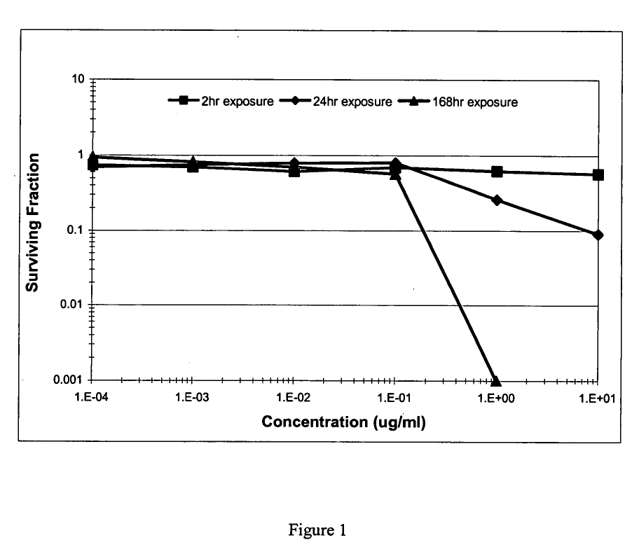

Data from the clonogenic assay with compound IV are shown in the FIG. 1.

These results for H116 human colon cells indicate that if concentrations of compound IV in the order of 10 μg/ml was maintained for a 24 h period; or, 0.2 μg/ml for a 7-day continuous exposure, a therapeutic effect would be obtained.

In Vivo Dose Confirmation Study

In this dose calculation stage, the projected starting dose is based primarily on the solubility of the new compound. Potential doses are calculated on the basis of blood volume and total body water in mice.

The starting dose is selected on the basis of solubility of the compound in biologically compatible solvents, and the potency derived from in vitro studies.

To start, one normal SCID mouse is dosed intravenously with the solubilized/formulated compound via a one-hour infusion at a maximum dose of 20 mg/mL. The volume infused is 0.25 ml over the one-hour period. If the animal survives the dosing period, clinical signs are noted, and the animal is sacrificed at two hours post infusion and blood collected for subsequent analytical determinations. Two additional SCID mice are then similarly infused with the compound, one being sacrificed at the end of infusion and blood collected and analyzed for drug concentration. The third animal is housed for 2 weeks to ascertain survival, and then euthanized. During the two-week observation period, the animal will be monitored for clinical signs of distress and euthanized if moribund. The dosing data and pharmacologic information (estimate of half-life) from these three animals will guide the subsequent time-course study by approximating a tolerable dose and clearance of the agent. If the first animal appears moribund during the dosing period (signs include labored breathing, tremors, convulsions and unconsciousness), it will be sacrificed and a second SCID mouse will be administered the agent at 50% the original dose via a 1 h intravenous infusion. If this animal survives, a second and third animal will be dosed as above with subsequent blood collection and analysis.

Lethal Dose for a bolus injection of compound IV was observed at 50 mg/kg. At 40 mg/kg the animals survived but looked shaky right after injection.

Pharmacokinetics

Compound IV was administered at 18.5 mg/kg to male SCID mice bearing H116 tumors. The pharmacology results indicated that approximately 1 μg/ml can be attained in the plasma and tumor over at least a 2-h period.

By 24 h after drug administration, the concentration of compound IV is about 100 ng/ml.

In Vivo Therapeutic Trials

For the in vivo studies HCT-116 cells in SCID mice will be used. Individual mouse body weights for each experiment are within 5 g and all mice are over 17 g at the start of therapy. The mice are supplied food and water ad libidum. The animals are pooled, implanted subcutaneously with 10⁷ cultured cells, and pooled again before distribution to treatment and control groups. Chemotherapy is started when the tumors reach a measurable size (about 150 mm³). Initially, a 1 hour intravenous infusion will be given and the whole animal toxicity and therapeutic effect correlated with the pharmacology studies described above. Dosage levels are usually closely spaced (2-fold increments or 0.5 decrements). All drugs will be administered intravenously. Mice are killed when their tumors reach 1500 mm³. Tumor weights are estimated from 2-dimensional caliper measurements done twice a week:
Tumor Weight (mg)=(a×b²)/2
where a and b are the tumor length and width, respectively, in mm.

For this in vivo study with compound IV, 4 mice were in the control group, 4 in the 2 mg/ml and 1 in the 3 mg/ml groups.

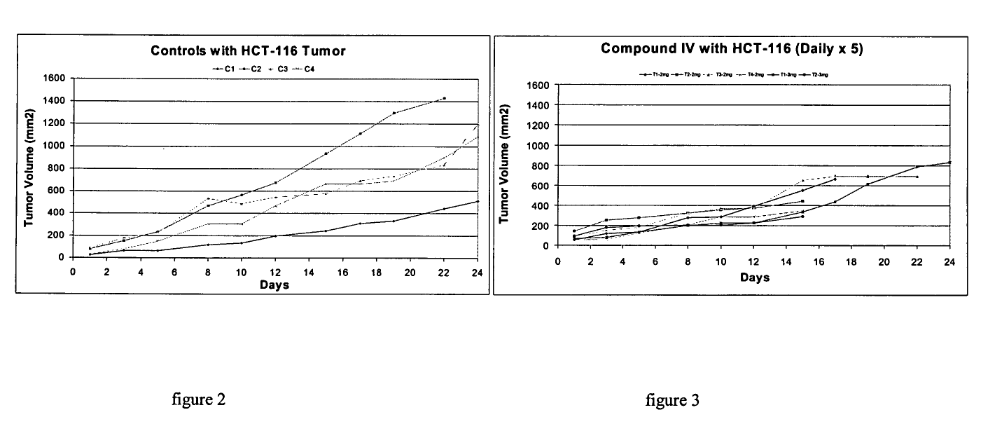

Data are shown in the FIGS. 2 and 3:

The data were analyzed in terms of time taken for tumor volume to reach 4-times the initial volume. For control tumors this was 7 days, for the 2 mg/ml (0.5 mg/mouse; 25 mg/kg) dose, this was 10 days; and for the 3 mg/ml (0.75 mg/mouse; 38.5 mg/kg), this was 15 days. Thus, at the maximum tolerated dose there was a T/C of about 50%.

1. A compound of general formula I or a pharmaceutically acceptable salt, derivative or prodrug thereof; 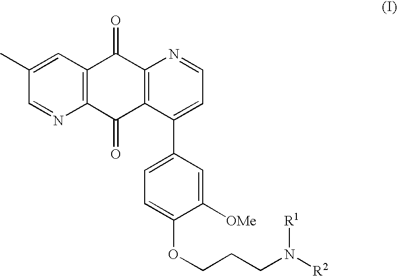
wherein R¹ and R² are independently selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₁-C₁₂ alkylidene, substituted or unsubstituted C₂-C₁₂ alkenylidene, substituted or unsubstituted C₃-C₁₂ alkynylidene and substituted or unsubstituted C₂-C₁₂ acyl:

2. A compound according to claim 1, which is of general formula II: 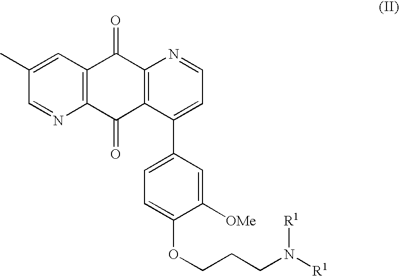
wherein R¹ is as defined in claim 1.

3. A compound according to claim 2, wherein R¹ is selected from the group consisting of H and C₁-C₁₂ alky.

4. A compound according to claim 1, which is of general formula III 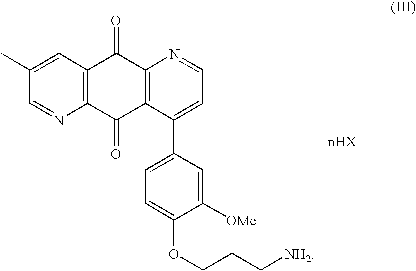
wherein X is selected from the group consisting of F, Cl, Br and I.

5. A compound according to claim 4 which is of formula IV:

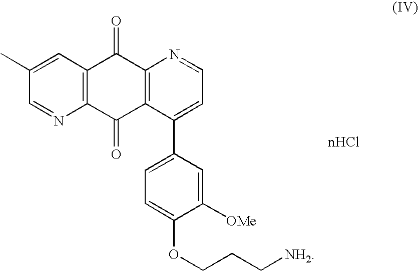

6. A pharmaceutical composition containing a compound of formula I or pharmaceutically acceptable salt, derivative or prodrug thereof;. 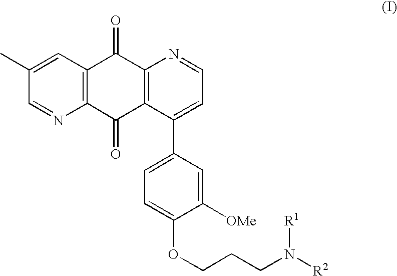
wherein R¹ and R² are independently selected from the group consisting of hydrogen, substituted or unsubstituted C₁-C₁₂ alky, substituted or unsubstituted C₂-C₁₂ alkenyl, substituted or unsubstituted C₂-C₁₂ alkynyl, substituted or unsubstituted C₁-C₁₂ alkylidene, substituted or unsubstituted C₂-C₁₂ alkenylidene, substituted or unsubstituted C₃-C₁₂ alkynylidene and substituted or unsubstituted C₂-C₁₂ acyl; together with a pharmaceutically acceptable carrier.

7. A method for the treatment of cancer which comprises administration of an effective amount of a compound of formula I or a pharmaceutically acceptable salt, derivative or prodrug thereof; 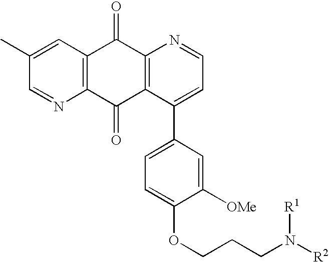
wherein R¹ and R² are independently selected from the group consisting of hydrogen, substituted or unsubstituted C₁-C₁₂ alky, substituted or unsubstituted C₂-C₁₂ alkenyl, substituted or unsubstituted C₂-C₁₂ alkynyl, substituted or unsubstituted C₁-C₁₂ alkylidene, substituted or unsubstituted C₂-C₁₂ alkenylidene, substituted or unsubstituted C₃-C₁₂ alkynylidene and substituted or unsubstituted C₂-C₁₂ acyl.

8. A process for preparing a compound of formula I or a pharmaceutically acceptable salt, derivative or prodrug thereof; 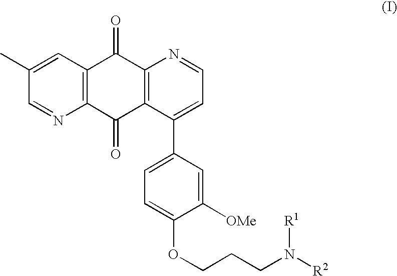
wherein R¹ and R² are independently selected from the group consisting of hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted C₁-C₁₂ alkylidene, substituted or unsubstituted C₂-C₁₂ alkenylidene, substituted or unsubstituted C₃-C₁₂ alkynylidene and substituted or unsubstituted C₂-C₁₂ acyl;
which process comprises reacting an N-protected {3-[2-methoxy-4-(4,4,5,5-tetramethyl-[1,3,2]dioxaborolan-2-yl)-phenoxy]-propyl}-carbamic acid of formula: 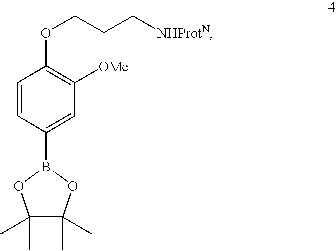
where Prot^{N} is a protecting group, with a 9-halo-3-methyl-pyrido[2,3-g]quinolin-5,10-dione of formula: 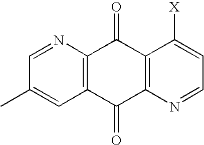
to give a protected form of a compound of this invention of formula: 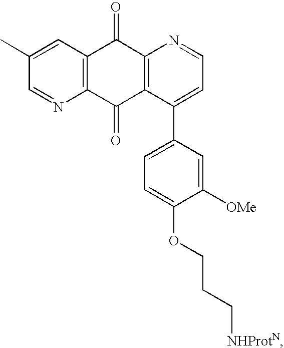
removing the protection, optionally introducing on the terminal amino group a substituent R¹ and/or R² when not hydrogen, and optionally salifying the compound to give a pharmaceutically acceptable salt.
